(19) United States
(12) Patent Application Publication
(10) Pub. No.: US 2021/0023023 A1
(43) Pub. Date: Jan. 28, 2021

(54) LIPOSOMAL NUTRACEUTICAL COMPOSITIONS AND METHODS OF MAKING THE SAME

(71) Applicant: VASAYO, LLC, Pleasant Grove, UT (US)

(72) Inventor: Tracy Gibbs, Herriman, UT (US)

(21) Appl. No.: 16/937,545

(22) Filed: Jul. 23, 2020

Related U.S. Application Data
(60) Provisional application No. 62/878,476, filed on Jul. 25, 2019.

(51) Int. Cl. A61K 31/05; A61K 9/127; A61K 36/752; A61K 36/53; A61K 47/10; A61K 47/44; A61K 38/48; A23L 33/105

(52) U.S. Cl. CPC A61K 31/05; A61K 9/1271; A61K 36/752; A61K 36/53; C12Y 304/22033; A61K 47/44; A61K 38/4873; A23L 33/105; C12Y 304/22002; A61K 47/10

(57) ABSTRACT

Disclosed are liposomal supplement compositions and methods of formulating the same. A liposomal composition includes a nutraceutical compound such as cannabidiol (CBD), a proteolytic enzyme component such as papain and/or bromelain, and a phytochemical component such as piceatannol. These components are encapsulated within liposomes for enhanced bioavailability and effectiveness.

### CROSS-REFERENCE TO RELATED APPLICATIONS

This application claims priority to and the benefit of U.S. Provisional Patent Application No. 62/878,476, filed Jul. 25, 2019 and titled “Liposomal Cannabidiol,” the entirety of which is incorporated herein by this reference.

### BACKGROUND

### Technical Field

This disclosure generally relates to liposomal supplement compositions formulated for enhanced bioavailability and effectiveness, and methods of formulating such liposomal supplement compositions. Particular aspects of the present disclosure relate to pain relief compositions including liposome-encapsulated cannabidiol (CBD) for the treatment of pain, and methods of manufacturing the same, among other exemplary liposome-encapsulated nutraceuticals.

### Related Technology

Opioids are a diverse class of relatively strong painkillers, including oxycodone, hydrocodone, and fentanyl. Fentanyl is synthesized to resemble other opiates such as opium-derived morphine and heroin. The potency and availability of these substances, despite their high risk of addiction and overdose, have made them popular as both medical treatments and as recreational drugs. However, due to their sedative effects on the part of the brain which regulates breathing, opioids in high doses may cause respiratory depression, which may lead to respiratory failure and death.

The addictive nature of opioids has led to an “opioid epidemic” in the United States, which refers to the rapid increase in the use of prescription and nonprescription opioid drugs. The U.S. has seen a dramatic increase in the number of opioid related deaths, with opioids being responsible for the majority of deaths due to drug overdose. Drug overdoses have become one of the major causes of death of Americans under 50.

In the late 1990s, around 100 million people or one-third of the U.S. population were estimated to be affected by chronic pain. This led to a nation-wide expansion in the use of painkilling opioids. Between 1991 and 2011 pain killer prescriptions in the U.S. tripled, and in 2016 more than 289 million prescriptions were written for opioid drugs.

The high death rate by overdose, the spread of communicable diseases, and the economic burden of the opioid crisis are major issues. A 2016 study showed the cost of prescription opioid overdoses, abuse and dependence in the U.S. in 2013 was approximately $78.5 billion, most of which was attributed to health care and criminal justice spending, along with lost productivity. Several studies have been conducted to find out how opioids were primarily acquired by opioid abusers. A 2013 national survey indicated that 74% opioid abusers acquired their opioids directly from a single doctor, or from a friend or relative who in turn received their opioids from a clinician.

Though patients are still in need of painkillers to treat chronic pain and post-operative pain, the destructive and addictive nature of opioids has led patients and clinicians alike to seek alternative pain management treatments. One of these pain management treatments is the use of cannabinoids.

Cannabinoids are diverse chemical compounds which act on cannabinoid receptors in the human endocannabinoid system. Exocannabinoids, referring to any cannabinoid not made within the body, include phytocannabinoids (found in pants) and synthetic cannabinoids (manufactured artificially). Many plants produce cannabinoids, but most people associate cannabinoids with the Cannabis sativa L. plant. Cannabinoids typically function in the human body by acting on CB1 and CB2 receptors in the brain and central nervous system, directly or indirectly, though other mechanisms of action may also be involved.

The most well-known cannabinoids are tetrahydrocannabinol (THC) and cannabidiol (CBD). THC is associated with the intoxicating “high” from cannabis consumption, while CBD is a non-intoxicating cannabinoid with analgesic properties.

Cannabinoids may be administered through a number of delivery means including topically (via topical creams, oils, salves, etc.), through inhalation (via vaporizing, smoking, etc.), orally (via sublingual drops or tablets or ingestion of foods with added cannabinoids called “edibles”).

The effectiveness of cannabinoids in treating specific conditions is depends at least in part on the route of administration. Different delivery routes may produce different therapeutic effects. For example, a topical application may provide localized relief, while a sublingual preparation may provide systemic, longer lasting effects.

In order to be a sufficient substitute for opioids in pain management, cannabinoid formulations should be quick-acting, long-lasting, and able to deliver a sufficient degree of pain relief. Though cannabinoids have been found to have some pain-relieving properties, cannabinoid formulations have thus far failed to deliver pain relief to a degree that would allow replacement or significant reductions in the use of opioids.

One issue with delivery of cannabinoids such as CBD is the bioavailability of the cannabinoid compound once administered to the user. For example, orally administered CBD is not readily absorbed when ingested and the majority will simply be excreted without exerting any effects to the user. Other supplements with potential benefits to users suffer from the same issues of poor bioavailability.

Accordingly, there is an ongoing need for supplement compositions that provide enhanced bioavailability and enhanced effectiveness when administered. This need is common to various different types of supplement compositions, including compositions that include CBD and are intended to provide effective pain relief.

### SUMMARY

A method of forming a liposomal supplement composition comprises the steps of: mixing oil-soluble ingredients (e.g., CBD and/or other oil soluble supplement) and a liposome subunit component (e.g., phosphatidylcholine) into an oil carrier (e.g., MCT oil); homogenizing the oil-soluble ingredients, the liposome subunit component, and the oil carrier to form an oil-based suspension; mixing water-soluble ingredients and a surfactant in an aqueous carrier; homogenizing the water-soluble ingredients with the surfactant and the aqueous carrier to form an aqueous suspension; mixing the homogenous oil-based suspension with the homogenous aqueous suspension to form a suspension of liposomal oil droplets stabilized by the surfactant and a suspension of droplets of the target supplement compound also stabilized by the surfactant; and cooling the mixed suspension to allow liposomes to capture the target supplement.

A liposomal cannabinoid composition comprises a cannabinoid (e.g., CBD) and one or more plant extracts encapsulated within liposomes. The plant extracts may comprise one or both of a proteolytic enzyme component or a phytochemical component. The composition may also include a preservative component associated with the structure of the liposomes themselves and/or generally mixed within the composition. Preferred proteolytic enzymes include bromelain and/or papain. The bromelain may be sourced from pineapple (e.g., fruit and/or other portions of the plant). The papain may be sourced from papaya (e.g., fruit and/or other portions of the plant). Preferred phytochemicals include piceatannol. The piceatannol may be sourced from passionfruit (e.g., fruit and/or other portions of the plant), such as from a passionfruit juice or concentrate. The pineapple, papaya, and/or passionfruit may be provided in the form of a juice (or concentrate provided at about 20-80 Brix), for example. Preferred preservative components include oregano oil and/or citrus oil (e.g., orange oil). The combined formulation may be encapsulated in a liposome that includes caprylic acid and/or other medium chain fatty acids, MCT oil, phosphatidylcholine, and optionally orange oil and/or oregano oil.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 is a flow chart illustrating a method for formulating a liposomal composition including one or more supplements encapsulated by liposomes;

FIGS. 2A and 2B graphically illustrate the expected improvement in bioavailability of an exemplary supplement composition (including CBD) resulting from liposomal encapsulation according to the method of FIG. 1, with FIG. 2A showing that peak absorption is expected to be increased and FIG. 2B showing that retention time is expected to be increased;

FIG. 3 graphically illustrates the expected improvement in bioavailability of another exemplary supplement composition (vitamin C) resulting from liposomal encapsulation according to the method of FIG. 1, showing greater peak concentration and area under the curve (AUC) of the liposomal vitamin C composition as compared to a standard (non-liposomal) vitamin C composition;

FIG. 4A illustrates a graph displaying the expected relative reduction in perceived pain among users administered (1) a placebo, (2) a liposomal formulation containing proteolytic enzymes and phytochemicals (without CBD), (3) a liposomal CBD formulation (without proteolytic enzymes or phytochemicals), or (4) a liposomal CBD formulation containing CBD, proteolytic enzymes, and phytochemicals according to one or more embodiments of the present disclosure;

FIG. 4B illustrates a graph of the expected relative perceived relief from pain episodes during the period of time immediately following pain onset when administered (1) a placebo, (2) a liposomal formulation containing proteolytic enzymes and phytochemicals (without CBD), (3) a liposomal CBD formulation (without proteolytic enzymes or phytochemicals), or (4) a liposomal CBD formulation containing CBD, proteolytic enzymes, and phytochemicals according to one or more formulations of the present disclosure; and

FIG. 5 illustrates a graph of the expected relative perceived pain relief from pain episodes during the period of time immediately following pain onset when administered (1) a placebo, (2) a non-liposomal formulation containing CBD, proteolytic enzymes, and phytochemicals, and (3) a liposomal CBD formulation containing proteolytic enzymes, and phytochemicals according to one or more formulations of the present disclosure.

### DETAILED DESCRIPTION

### I. Introduction

Liposomal compositions that may be formulated using the methods described herein include a supplement component and a liposome component. The supplement component may include one or more vitamins, minerals, nutrients, herbs, amino acids, peptides, fatty acids, fatty acid esters, antioxidants, saccharides, ketone bodies, or other nutraceutically acceptable compounds suitable for administration to a user for the purpose of supplementing health. Specific examples include vitamin C, vitamin D3, vitamin K2, vitamin B12 or other B vitamins, omega 3 oils, glutathione, turmeric/curcumin, coenzyme Q10, resveratrol, carnosine, magnesium, quercetin, boswellia extract (AKBA), melatonin, and GABA. The terms “supplement” and “nutraceutical” are used interchangeably herein.

The following description and examples will focus mostly on liposomal compositions including CBD as the sole or major supplement component. However, the liposomal compositions described herein are not limited to those that include CBD, and additional or alternative supplement components may be included. Similarly, although pain-relief is a major purpose of some of the described examples, including those that comprise CBD, other embodiments may relate to additional or alternative uses and intended effects.

The supplement component is encapsulated in the liposome component in order to enhance the bioavailability and/or other pharmacokinetic properties of the supplement component when administered to the user. The term “administration” includes self-administration as well as administration from another. The liposomal composition may be provided in various dosage forms. Preferably, the liposomal composition is a gel or liquid, and may be provided in a dosage form suitable for gels or liquids, such as a tincture or dropper bottle, capsule, concentrate, or the like.

Some compositions include, in addition to the liposome-encapsulated supplement component, one or more proteolytic enzymes and/or phytochemicals. These added components may be encapsulated in the liposome along with the supplement component and/or may be un-encapsulated and merely mixed with the liposome-encapsulated supplement composition.

The term “proteolytic enzyme” and related terms are used herein to refer to enzymes that function to break the long chainlike molecules of proteins into shorter peptide fragments and/or into their amino acid subcomponents. Proteolytic enzymes described herein may be provided from plant sources. Examples include bromelain (e.g., sourced from pineapples), papain (e.g., sourced from papayas), though other proteolytic enzymes may additionally or alternatively be utilized, as described in more detail below.

The term “phytochemicals” and related terms are used herein to refer to organic compounds produced by plants and that show biological activity. Examples include: terpenes/terpenoids such as carotenoids; polyphenols (e.g., flavonoids, stilbenes) and other phenolic compounds; glucosinolates such as isothiocyanates and indoles; betalains; anthocyanins; and proanthocyanidins. Specific examples include quercetin, lutein, zeaxanthin, and stilbenes such as piceatannol and resveratrol.

Terms such as “user,” “patient,” “subject,” and the like are used interchangeably herein depending on context. Differing use of these terms is not intended to signify any particular subset of uses or applications unless specified as such.

### II. Liposomes & Methods of Manufacturing Liposomal Compositions

Generally, liposomes are formulated using polyethylene glycol (PEG) ingredients. Though PEG ingredients are generally considered biologically inert and safe, a portion of the population has been found to be allergic or otherwise sensitive to PEG ingredients and compositions formed via PEGylation. Furthermore, when PEG is chemically attached to therapeutic molecules, such as protein drugs or nanoparticles, it can sometimes be antigenic, stimulating an anti-PEG response in some users.

Alcohol ingredients are also conventionally used in the formation of liposomes for delivery of a target compound. Liposomes incorporating alcohol and/or PEG ingredients may stifle the pharmacokinetics or overall effectiveness of the resulting liposomal composition, and preferred embodiments of liposomal compositions described herein are beneficially formulated without requiring PEG or alcohol.

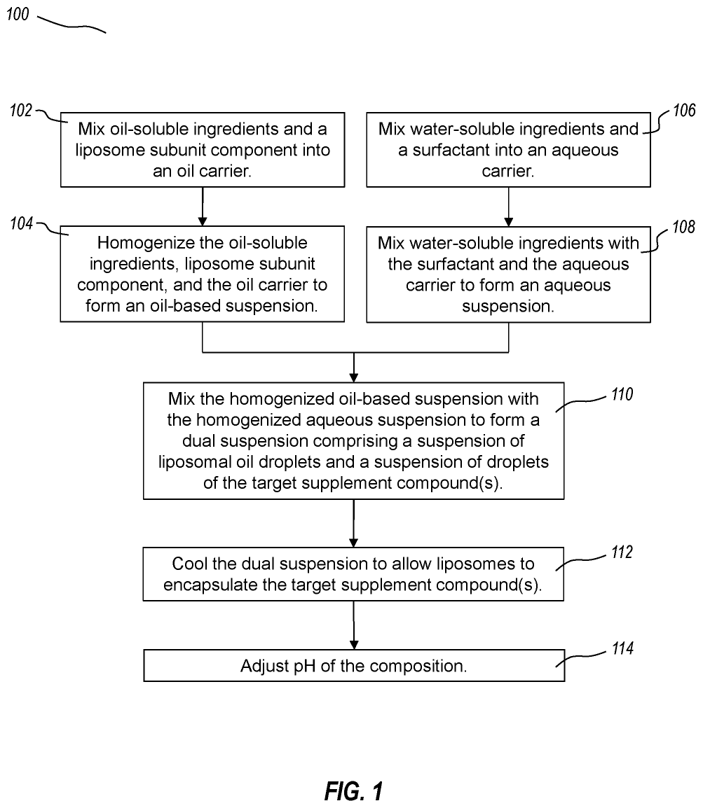

FIG. 1 illustrates a method 100 for forming a liposomal composition. As shown, the method 100 includes a step 102 of mixing oil-soluble (used synonymously herein with “fat-soluble”) ingredients and a liposome subunit component into an oil carrier. The oil carrier preferably comprises or essentially consists of medium chain fatty acids or esters thereof (e.g., caprylic acid, decanoic acid, lauric acid, and their esters), such as medium chain triglyceride (MCT) oil. For example, the oil carrier may comprise or consist essentially of coconut (Cocos nucifera) oil. Although many types of oil carriers may be utilized, it has been found that MCT oil derived from coconuts provides more effective structural integrity to the resulting liposomes as well as providing its own independent health benefits.

The oil-soluble ingredients may include, for example, any supplement components targeted for encapsulation where those supplements are fat/oil-soluble (such as the case with CBD). Other oil-soluble ingredients may include preservatives (e.g., oregano oil and/or citrus oil), which can beneficially function to sterilize the and/or enhance the stability of the subsequently formed liposomes.

The liposome subunit component includes one or more compounds capable of forming liposomes. Preferred examples include nutraceutically acceptable phospholipids such as phosphatidylcholine and phosphatidylethanolamine. Phosphatidylcholine, when included, may be derived from natural sources such as sunflower (Helianthus annuus) oil.

The method 100 also includes a step 104 of homogenizing the oil-soluble ingredients, the liposome subunit component, and the oil carrier to form an oil-based suspension. The homogenization may be carried out using a high-speed homogenization system. The system may mix the ingredients at speeds of 8,000 RPM to 30,000 RPM, more preferably from 10,000 RPM to 25,000 RPM or from 12,000 RPM to 20,000 RPM, for about 10 to 35 minutes, more preferably for about 12 to 30 minutes or more preferably about 15 to 25 minutes.

During the homogenization step 104, the fat-soluble ingredients may be mixed with the oil carrier at a temperature of about 42° C. to 55° C., or more preferably about 45° C. to 50° C., by the high-speed homogenization system until the fluid appears homogenous. Homogenization of these components at mixing speeds, durations, and/or temperatures within the foregoing ranges has been found to promote good results in subsequent liposome formation steps (e.g., by promoting desired liposome characteristics and/or raising encapsulation efficiency).

The method 100 also includes a step 106 of mixing water-soluble ingredients and a surfactant in an aqueous carrier (e.g., water or purified water). The surfactant may comprise or consist essentially of a polyethoxylated oil (e.g., Cremophor® EL, also known as Kolliphor® EL). The water-soluble ingredients may include, for example, any supplement components that are water soluble.

The method 100 also includes a step 108 of homogenizing the water-soluble ingredients with the surfactant and the aqueous carrier to form an aqueous suspension. These components may be mixed using a high-speed homogenization system at speeds and with a duration similar to those described above for the oil-soluble ingredients. The water-soluble ingredients may be mixed with the surfactant at 40° C. to 50° C., more preferably 45° C. to 55° C., until the solution appears homogenous.

The temperature may be the same as with the homogenization of the fat-soluble ingredients, but it has been determined that end results are improved when the temperature is slightly higher than during homogenization of the oil-soluble ingredients, such as by about 2° C. to 8° C. higher. Homogenization of these components at mixing speeds, durations, and/or temperatures within the foregoing ranges has been found to promote good results in subsequent liposome formation steps (e.g., by promoting desired liposome characteristics and/or raising encapsulation efficiency).

The method 100 then proceeds with a step 110 of mixing the homogenous oil-based suspension with the homogenous aqueous suspension. This results in a dual suspension comprising a suspension of liposomal oil droplets stabilized by the surfactant and a suspension of particles/droplets of the target supplement compound(s) also stabilized by the surfactant.

The method 100 proceeds with a step 112 of cooling the dual suspension. The cooling step preferably lasts about 12 to about 36 hours (e.g., about 24 hours). During cooling, the liposomes form around and encapsulate the droplets of the target supplement compounds, forming a stable liposome with a high encapsulation efficiency (EE). The method 100 described herein has been shown to beneficially provide an EE of 60% or higher, and more typically about 80% or higher.

Optionally, for additional stability, the method 100 may include a step 114 of adjusting the pH of the composition. The pH may be tested to determine whether the pH of the composition is greater than 4.8, more preferably greater than 5.0 or greater than 5.3. At pH levels higher than these thresholds, the liposomal wall is more likely to remain intact. The pH may be brought closer to neutral pH, if desired, by adding phosphate-buffered saline (PBS) and/or other suitable buffer.

The particle size of the liposomes is preferably between about 200 nm to about 450 nm, or about 250 nm to about 400 nm. The particle size may be measured using techniques known in the art (e.g., using a Malvern Zetasizer®) to find the Z average value. Particle size may be measured again after 48 hours to determine whether the particle size has remained substantially stable, thereby signaling the formation of a stable liposome.

The liposome encapsulation of the supplement compound(s) can beneficially enhance the therapeutic effect of the composition for the user, such as by enhancing bioavailability, absorption, area under the curve (AUC), Cₘₐₓ, or other pharmacokinetic properties of the composition. Liposomes form a lipid bilayer, closely resembling the structure of cell membranes. Many supplement compounds, including CBD, are predominantly fat-soluble, and the liposome may be advantageously used as a delivery vehicle.

Effective liposome formation results have been found using ingredients in amount according to one or more of: caprylic acid at about 0.25-3 wt. %, or more preferably about 0.6-1.8 wt. %; MCT oil at about 0.25-3 wt. %, or more preferably about 0.5-1.5 wt. %; phosphatidylcholine at about 0.25-3 wt. %, or more preferably at about 0.9-1.5 wt. %; citrus oil at about 0.001-0.012 wt. %, or more preferably about 0.004-0.009 wt. %; and oregano oil at about 0.001-0.012 wt. %, or more preferably about 0.004-0.009 wt. %. In the foregoing, the wt. % amounts refer to percentage of the entire liposomal composition after all ingredients are mixed.

### III. Overview of Liposomal Cannabinoid Compositions

Although the present disclosure relates to liposomal compositions for a variety of different supplements, the following description focuses particularly on an embodiment of a liposomal supplement composition comprising CBD as active ingredient. Embodiments that include other supplements in addition to CBD or as an alternative to CBD may still utilize any combination of the other ingredients (e.g., phytochemicals, proteolytic enzymes, preservatives, plant extracts) descried in relation to the liposomal CBD composition below.

In an exemplary embodiment, a liposomal cannabinoid composition comprises a cannabinoid (e.g., CBD) and one or more plant extracts encapsulated within liposomes. The plant extracts may comprise one or both of a proteolytic enzyme component or a phytochemical component. The composition may also include a preservative component associated with the structure of the liposomes themselves and/or generally mixed within the composition.

A. Cannabinoids

In some embodiments, the liposomal cannabinoid composition may contain between 0.0-0.3 wt. % THC. In some embodiments it may be preferred to remove all THC from the composition. In other embodiments it may be preferred that the CBD contain between 0.001-0.3 wt. % THC, which is usually considered an insignificant amount not worth extra processing for removal. Other embodiments may include more than 0.3 wt. % THC.

In some embodiments, the liposomal CBD formulation may incorporate a particular amount of THC, which may be naturally occurring in the particular plant source from which the CBD is derived. Alternatively, a particular amount of THC may be added to activate the agonist-antagonist synergistic effects caused by particular ratios of CBD:THC. The synergistic ratio of CBD:THC may reinforce the therapeutic effects of the other active ingredients in the formulation.

One or more other cannabinoid compounds may additionally or alternatively be included. The cannabinoid compound(s), and in particular CBD, may be included at 0.1-2.0 wt. %, more preferably 0.2-1.5 wt. %, more preferably 0.3-0.9 wt. %.

Plant cannabinoids have been shown to effectively treat pain related to a number of conditions including chronic pain and inflammation. CBD is a plant cannabinoid derived from the Cannabis sativa L. plant. The term “CBD,” as used herein is intended to be understood as cannabidiol derived from hemp varieties of Cannabis sativa L. or related cannabis species containing less than 0.3 percent THC, as well as cannabidiol derived from varieties of Cannabis sativa L. or related cannabis species containing up to or more than 0.3 percent THC.

The therapeutic properties of CBD may be beneficial for a wide variety of health issues, including pediatric epilepsy, anxiety, and insomnia. CBD may be used to treat chronic pain and inflammation such as pain and inflammation associated with arthritis. CBD is practically non-psychotropic and may counteract the cognitive impairment associated with the use of THC. CBD is not believed to bind with the CB1 and CB2 receptors but rather acts as an indirect antagonist of other cannabinoid agents. For example, in contrast to THC's agonist activity of CB1 receptors, CBD may act as an allosteric modulator of the CB1 receptor. At the same time, CBD may potentiate the effects of THC by increasing CB1 receptor density or through other CB1 receptor-related mechanisms.

CBD has powerful anti-inflammatory and analgesic effects mediated by both cyclooxygenase and lipoxygenase inhibition. In studies, CBD has been shown to have anti-inflammatory effects several hundred times more potent than aspirin. Research suggests that CBD may achieve its pharmacological effects by inhibition of fatty acid amide hydrolase (FAAH), which may in turn increase the levels of endocannabinoids produced by the body. It has also been speculated that some of the metabolites of CBD have pharmacological effects that contribute to the biological activity of CBD. There is medical evidence showing that CBD may be effective in the treatment of a wide range of disorders.

CBD administered alone has been found to result in moderate pain reduction. However, conventional CBD formulations do not deliver pain relief reliably enough or at levels sufficient to realistically function as a widespread opioid replacement.

B. Proteolytic Enzymes

Proteolytic enzymes are produced in the human body as well as by various plants. In the human body, proteolytic enzymes are commonly known for their role in digestive processes. However, proteolytic enzymes may be essential for other processes such as cell division, blood dotting, immune function, and protein recycling. Proteolytic enzymes found in plants include endopeptidases, dipeptidyl peptidases, and enzymes with both exopeptidase and endopeptidase activity.

Some of the best food sources of proteolytic enzymes are papaya and pineapple. Papayas (Caricaceae family) contain an enzyme called papain or papaya proteinase I. Papain is found in the leaves, roots and fruit of the papaya plant. Papain is at times used as a meat tenderizer due to its ability to break down protein. This enzyme may act as an anti-inflammatory and analgesic agent. It may also act as a digestive aid.

Bromelain is found in Bromeliaceae such as in the fruit skin and juice of the pineapple plant (Ananas comosus). Like papain, bromelain has a history of use as a meat tenderizing ingredient and as a digestive aid. Bromelain may be effective as an antithrombotic and anti-inflammatory agent, and has been studied in association with treatments for osteoporosis, asthma, chronic sinusitis, colitis, burns, and cancer. Bromelain may reduce C-reactive protein (CRP) levels and other markers for inflammation such as fibrinogen.

Though pineapples and papayas are the most common sources of proteolytic enzymes, other dietary sources may include kiwi fruit, ginger, asparagus, sauerkraut, kimchi, yogurt, and kefir. Proteolytic enzymes may also be derived from animal sources, such as trypsin and chymotrypsin, which are typically derived from pigs and cows. Enzyme activity levels may be reported in Food Chemical Codex (FCC) units. Protease enzymes may be commonly labeled in HUT (Hemoglobin Unit Tyrosine base), or USP (United States Pharmacopeia) units, where 1 HUT equals approximately 6.5 USP.

Where papain is provided via papaya juice and/or bromelain is provided via pineapple juice, the juice may be provided at about 10-30 wt. %, or more preferably about 14-22 wt. %. Where papain and/or bromelain are provided through another form (e.g., a concentrate), the other form may be provided in amounts so as to provide a “papain equivalent” or “bromelain equivalent” similar to when the foregoing juice amounts are utilized.

C. Phytochemicals

Liposomal supplement compositions such as a liposomal CBD composition may also include one or more phytochemicals. Examples include: terpenes/terpenoids such as carotenoids; polyphenols (e.g., flavonoids, stilbenes) and other phenolic compounds; glucosinolates such as isothiocyanates and indoles; betalains; anthocyanins; and proanthocyanidins.

One particularly preferred phytochemical is piceatannol. Piceatannol is a stilbenoid found in mycorrhizal and non-mycorrhizal roots of Norway spruces (Picea abies). It can also be found in the seeds of the palm Aiphanes horrida and in Gnetum cleistostachyum, and is abundant in the seeds of passionfruit (Passiflora edulis). The chemical structure of piceatannol is a known analog of resveratrol. Piceatannol has been found to alter the timing of gene expressions, gene functions, and insulin action, resulting in the delay or complete inhibition of adipogenesis. Piceatannol may also act as an insulin sensitivity enhancer. There is a correlation between chronic pain and insulin resistance, and acute pain is known to induce insulin resistance.

Where piceatannol is provided via passionfruit, the passionfruit may be provided as a juice (non-concentrate) at about 10-30 wt. %, more preferably about 15-25 wt. %, or more preferably about 18-22 wt. %. Where piceatannol is provided through another form (e.g., a concentrate), the other form may be provided in amounts so as to provide a “piceatannol equivalent” similar to when the foregoing juice amounts are utilized.

D. Preservatives

The liposomal compositions such as the liposomal CBD formulation may include preservatives. For example, the liposomal composition may be formulated with one or more essential oils. The preservative oils may include one or more oils of culinary herbs, such as oregano oil. The preservative oils may additionally or alternatively include one or more citrus oils, such as orange oil.

The preservative ingredients may provide a sterilizing mechanism in the liposome creation process. Furthermore, the preservative ingredients may preserve the integrity of the liposome which ensures enhanced bioavailability of the liposomal composition.

E. Additional Ingredients

Other ingredients may include one or more of various excipients, distilled water, floral extract such as hibiscus (flower) infused floral water, vitamin C, and/or vegetable glycerin. The floral extract and/or vitamin C may impart the liposomal composition with antioxidant properties which may improve the shelf life of the formulation and impart the formulation with desirable flavor properties.

The liposomal composition may include one or more of: distilled water at 10-20 wt. %, or more preferably 13-18 wt. %; hibiscus-infused floral water at 10-20 wt. %, or more preferably 13-18 wt. %; vitamin C at 1.0-5.0 wt. %, or more preferably 2.0-4.0 wt. %; or vegetable glycerin at 1.0-5.0 wt. %, or about 2.0-4.0 wt. %.

### IV. Exemplary Effects

Liposomal cannabinoid compositions enable treatment of pain associated with, for example, chronic pain, post-surgical pain, inflammation, arthritic pain, pain from injuries, post-operative recovery, and internal inflammatory conditions such as fibromyalgia and lupus. Beneficial analgesic effects are provided through the unexpected synergistic effect of a combination of CBD with one or more of the plant extracts described above, and through the enhanced pharmacokinetics provided by effective liposome encapsulation of the active ingredients. As described above, the liposomes may include phosphatidycholine, caprylic acid and/or other medium chain fatty acids, MCT oil, and optionally orange oil and/or oregano oil.

CBD, bromelain, papain, and piceatannol may each individually provide some amount of health benefits. However, individually, they do not provide a degree of pain relief close to the relief provided by opioid painkillers. Surprisingly, when CBD, bromelain, papain and piceatannol are combined, the pain-relieving capabilities of the combined formulation are unexpectedly found to be much higher than would be expected based on the additive, individual effects of each component. In at least some applications, it is believed that the analgesic effect of the combination may be at least effective enough to allow for a replacement or significant reduction of opioid painkillers commonly prescribed for similar ailments such as chronic pain and post-surgical pain.

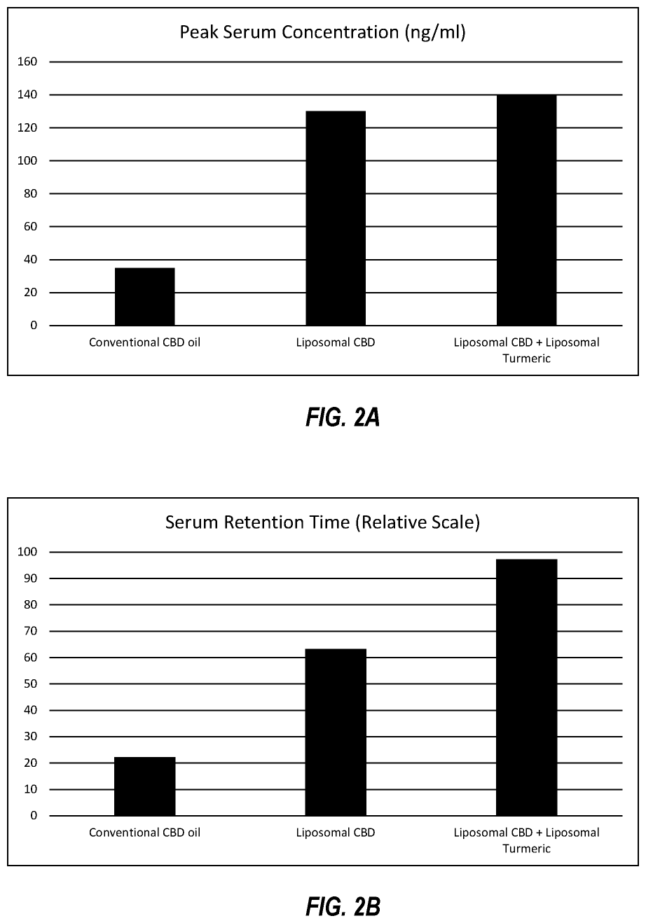

FIGS. 2A and 2B graphically illustrate the expected (prophetic) improvement in bioavailability of an exemplary liposomal CBD composition resulting from liposomal encapsulation according to the method of FIG. 1, with FIG. 2A showing that peak absorption is expected to be increased and FIG. 2B showing that retention time is expected to be increased. As shown, the liposomal CBD formulation is expected to provide greater peak serum concentration and greater serum retention time as compared to conventional (non-liposomal) CBD oil.

FIGS. 2A and 2B also illustrate that the bioavailability of one supplement type may be enhanced by the inclusion of another supplement type. In this case, it is expected that the bioavailability of CBD in the liposomal CBD formulation may be further enhanced with the inclusion of liposomal turmeric (or isolated curcumin that has been encapsulated in liposomes).

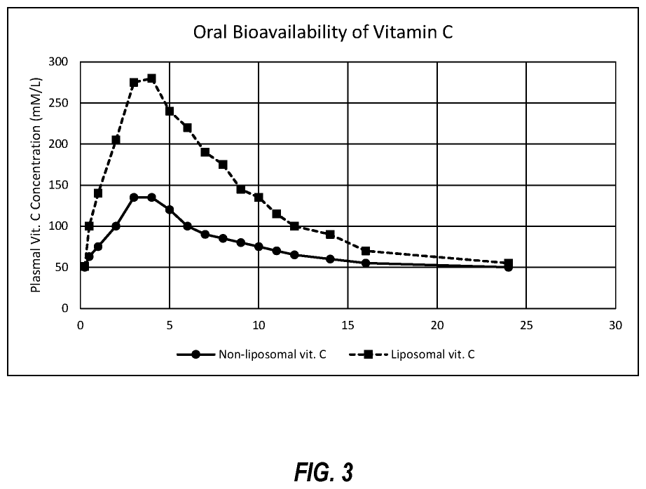

FIG. 3 illustrates the expected improvement in bioavailability of another exemplary supplement composition (vitamin C) resulting from liposomal encapsulation according to the method of FIG. 1, showing greater peak concentration and area under the curve (AUC) of the liposomal vitamin C composition as compared to a standard (non-liposomal) vitamin C composition. Liposomal encapsulation may also enable greater effectiveness for topical administration of supplement types amenable to topical administration, such as vitamin C.

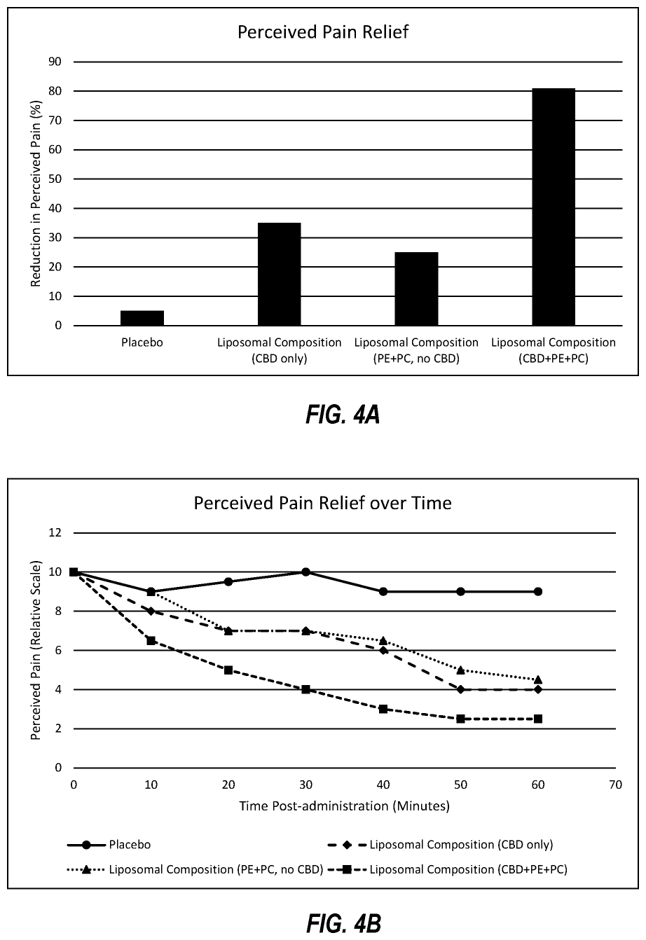

FIGS. 4A and 4B illustrate expected results of a prophetic comparative study. The study may be carried out by organizing groups of individuals experiencing pain and treating the separate groups with (1) a placebo, (2) a liposomal formulation containing CBD but not containing proteolytic enzymes or phytochemicals, (3) a liposomal formulation containing proteolytic enzymes and phytochemicals but not CBD (PE+PC, no CBD), or (4) a liposomal formulation containing CBD, proteolytic enzymes, and phytochemicals (CBD+PE+PC).

FIG. 4A may be understood to describe the expected relative reduction in pain perceived by individuals experiencing pain due to chronic pain or pain related to having undergone a surgical procedure, for example. The study may be carried out by establishing a baseline for perceived level of pain for all treatment groups, which may be done by tracking the pain level experienced over a time period equal to the testing period before the testing phase begins. Pain level may be calculated based on the frequency of pain experienced by the individual and the severity of the pain during each episode of pain, such as through a subjective 0-10 scale pain rating reported by each of the subjects over time.

As an example of how such a study may be carried out, an individual in a one-week trial first tracks his/her pain level for one week prior to the beginning of the study. Similarly, an individual in a two-week study tracks their pain level for two weeks prior to beginning the trial. The groups may be related to a specific dosage period. For example, group 1 individuals may use their respective treatments for a one week period and the level of pain experienced per day is reported daily and resulting pain level experienced is calculated at the end of the week. The group 2 individuals may use their respective treatments for a period of at least two weeks and up to one month. Or, in some implementations, the group is pre-treated for 1-2 months prior to the data collection period so that the liposomal composition has been able to build up the therapeutically relevant levels and/or to have a relevant physiological effect on the individual.

In some implementations, the frequency and severity at which an individual experiences pain is recorded daily, and the resulting pain level is calculated at the end of the trial period. The group 3 individuals may use their respective treatments for a period of at least one month and up to three months. During this period the pain level is recorded daily. group 4 individuals may use their respective treatments for at least three months and up to six months. The pain level is recorded on a daily basis, and the resulting pain level is calculated at the conclusion of the trial period.

The expected reduction in pain level as portrayed in FIG. 4A demonstrates a reduction in pain level across subjects treated with the liposomal formulation including CBD but not including proteolytic enzymes or phytochemicals (CBD only). Groups treated with the liposomal formulation without CBD (PE+PC, no CBD) also experience a reduction in pain level as compared to the placebo group. The individuals treated with a liposomal CBD formulation containing CBD, proteolytic enzymes and phytochemicals (CBD+PE+PC) experience a substantially greater decrease in pain level as compared to placebo and the other groups.

FIG. 4B depicts expected perceived relief over time in response to the administration of formulations for a particular pain attack or episode. As with the prophetic example of FIG. 4A, individuals the separate treatment groups are given: (1) a placebo, (2) a liposomal CBD formulation containing CBD but not containing proteolytic enzymes or phytochemicals (CBD only), (3) a liposomal formulation containing proteolytic enzymes and phytochemicals but no CBD (PE+PC, no CBD), or a liposomal formulation containing CBD, proteolytic enzymes, and phytochemicals (CBD+PE+PC).

The study may be carried out by administering the respective treatment upon the onset on the pain attack or episode. The individual is monitored for a period of time following the initial dosage of a particular formulation. The individual records their perceived pain level at discrete time intervals, such as at 10-minute intervals over the course of 60 minutes. This study may be extended to account for pain lasting longer than 60 minutes and/or to observe the impact of a specific treatment over an extended period of time. Within the first 10-minutes post-administration of a particular formulation, the individual reports their present level of perceived pain on a scale of 0-10 with 0 representing negligible or no pain, and with 10 being the most severe pain. This process may be repeated at 10-minute intervals (or other suitable intervals) to observe a response to treatment.

Patients who are administered the liposomal CBD composition with proteolytic enzymes and phytochemicals (CBD+PE+PC) are expected to reach a perceived pain level lower than those associated with the other treatments. Patients who are administered the liposomal formulation with proteolytic enzymes and phytochemicals (PE+PC, no CBD), or who are administered the liposomal composition (CBD only) are expected to take longer to reach similar pain reductions, or to have less overall efficacy.

Ingested cannabinoids tend to require an extended period of time, typically at least 1-3 hours, in order for their effects to be felt by the individual. In the formulation of the present disclosure, the cannabinoids are delivered in a liposomal form that beneficially enhances absorption. The cannabinoids are also believed to interact with the proteolytic enzymes and phytochemicals in a manner that enhances and/or quickens the therapeutic effect of the formulation. These effects allow the patient to experience substantial relief in less time after the initial dosage is administered to treat pain such as chronic pain or post-surgery pain.

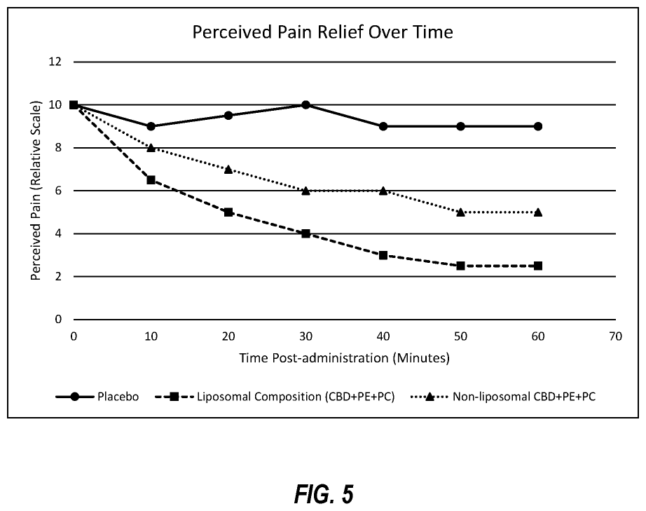

FIG. 5 illustrates the expected effect of liposomal encapsulation on the rate of absorption and resulting pain relief as compared to an otherwise similar formulation containing CBD, proteolytic enzymes (papain, bromelain), and phytochemicals (piceatannol), but not delivered via a liposome. FIG. 5 is a prophetic example, and in some applications the synergistic effects of the liposomal CBD formulation may take a longer period of time for the noticeable effect to occur. This may be due to specific physiological conditions of the subject, for example.

### V. Additional Formulation Details & Options

Pharmaceutical compositions (used interchangeably with “nutraceutical formulations”) useful in the methods and uses of the disclosed embodiments are provided. A pharmaceutical composition is any composition that may be administered to a subject to treat or ameliorate a condition. A subject may include an animal. In some embodiments, the animal is a mammal. The mammal may be a human or primate in some embodiments.

As used herein the terms “nutraceutically acceptable,” “pharmaceutically acceptable” and “physiologically acceptable” mean a biologically compatible formulation, gaseous, liquid or solid, or mixture thereof, which is suitable for one or more routes of administration, in vivo delivery, or contact. A formulation is compatible in that it does not destroy activity of an active ingredient therein or induce adverse side effects that outweigh any prophylactic or therapeutic effect or benefit.

In an embodiment, the pharmaceutical compositions may be formulated with pharmaceutically acceptable excipients such as carriers, solvents, stabilizers, adjuvants, diluents, etc., depending upon the particular mode of administration and dosage form. Formulations, for example, for parenteral or oral administration, are most typically solids, liquid solutions, emulsions or suspensions, while inhalable formulations for pulmonary administration are generally liquids or powders, with powder formulations being generally preferred. A preferred pharmaceutical composition may also be formulated as a lyophilized solid that is reconstituted with a physiologically compatible solvent prior to administration. Alternative pharmaceutical compositions may be formulated as syrups, creams, ointments, tablets, suppositories, transdermal patches, and the like.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin, or olive oil. In another embodiment, pharmaceutical compositions may be formulated as suspensions comprising a compound of the embodiments in admixture with at least one pharmaceutically acceptable excipient suitable for the manufacture of a suspension. In yet another embodiment, pharmaceutical compositions may be formulated as dispersible powders and granules suitable for preparation of a suspension by the addition of suitable excipients.

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing, or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g., dextran sulfate); glycoaminoglycans and glycosaminoglycan-like compounds (e.g., hyaluronic acid); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

A pharmaceutical composition and/or formulation contains a total amount of the active ingredient(s) sufficient to achieve an intended therapeutic effect. The pharmaceutical compositions may, for convenience, be prepared or provided as a unit dosage form. A “unit dosage form” as used herein refers to a physically discrete unit suited as unitary dosages for the subject to be treated; each unit containing a predetermined quantity of compound optionally in association with a pharmaceutical carrier (e.g., excipient, diluent, vehicle or filling agent) which, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound. Unit dosage forms also include, for example, capsules, troches, cachets, lozenges, tablets, ampules and vials. Unit dosage forms additionally include, for example, ampules and vials with liquid compositions disposed therein. The individual unit dosage forms can be included in multi-dose kits or containers. Pharmaceutical formulations can be packaged in single or multiple unit dosage forms for ease of administration and uniformity of dosage.

Compounds can be administered in accordance with the methods at any frequency as a single bolus or multiple dose e.g., one, two, three, four, five, or more times hourly, daily, weekly, monthly, or annually or between about 1 to 10 days, weeks, months, or for as long as appropriate. Exemplary frequencies are typically from 1-7 times, 1-5 times, 1-3 times, 2 times or once, daily, weekly or monthly. For example, twice weekly for two weeks. The dosage may range broadly, depending upon the desired effects and the therapeutic indication. Alternatively, dosages may be based and calculated upon the per unit weight of the patient, as understood by those of skill in the art. Although the exact dosage will be determined on a drug-by-drug or supplement-by-supplement basis, in most cases, some generalizations regarding the dosage can be made. The systemic daily dosage regimen for an adult human patient may be, for example, an oral dose of between 0.01 mg and 3000 mg of the active ingredient, preferably between 1 mg and 700 mg, e.g. 5 to 200 mg. In some embodiments, the daily dosage regimen is 1 mg, 5 mg, 10, mg, 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, or about any of the aforementioned numbers or a range bounded by any two of the aforementioned numbers.

### VI. Conclusion

It should be understood that for any given element of component of a described embodiment, any of the possible alternatives listed for that element or component may generally be used individually or in combination with one another, unless implicitly or explicitly stated otherwise. It will also be appreciated that embodiments described herein may include properties, features (e.g., ingredients, components, members, elements, parts, and/or portions) described in other embodiments described herein. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” “essentially,” or the like are used in conjunction with a stated amount, value, or condition, it may be taken to mean an amount, value or condition that deviates by less than 20%, less than 10%, less than 5%, or less than 1% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims. It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

1. A method of forming a liposomal composition comprising liposomes and a target compound encapsulated by the liposomes, the method comprising:
mixing oil-soluble ingredients and a liposome subunit component into an oil carrier;
homogenizing the oil-soluble ingredients, liposome subunit component, and the oil carrier to form an oil-based suspension;
mixing water-soluble ingredients and a surfactant into an aqueous carrier;
mixing the water-soluble ingredients with the surfactant and the aqueous carrier to form an aqueous suspension;
mixing the homogenized oil-based suspension with the homogenized aqueous suspension to form a dual suspension comprising a suspension of liposomal oil droplets and a suspension of droplets containing the target compound; and
cooling the suspension to allow liposomes to encapsulate the target compound and form the liposomal composition.

2. The method of claim 1, wherein the method omits the addition of polyethylene glycol (PEG).

3. The method of claim 1, wherein the method omits the addition of alcohol.

4. The method of claim 1, wherein the oil carrier includes medium chain triglyceride (MCT) oil.

5. The method of claim 1, wherein the oil-soluble ingredients comprise one or more plant essential oils as a preservative.

6. The method of claim 5, wherein the preservatives include citrus oil, oregano oil, or both.

7. The method of claim 1, wherein the liposome subunit component includes phosphatidylcholine.

8. The method of claim 1, wherein the surfactant includes a polyethoxylated oil.

9. The method of claim 1, wherein cooling is carried out for about 12 to about 36 hours.

10. The method of claim 1, further comprising a step of adjusting the pH of the liposomal composition so that the pH is greater than 4.8.

11. The method of claim 1, wherein the liposomes have an average particle size of about 200 nm to about 450 nm.

12. The method of claim 1, wherein the liposomes are formed with an encapsulation efficiency of 60% or higher.

13. The method of claim 1, wherein the target compound is a cannabinoid.

14. The method of claim 13, wherein the cannabinoid is cannabidiol (CBD).

15. A liposomal composition, comprising:
a nutraceutical compound;
a proteolytic enzyme component;
a phytochemical component; and
a plurality of liposomes encapsulating the nutraceutical compound, proteolytic enzyme component, and phytochemical component.

16. The liposomal composition of claim 15, wherein the nutraceutical compound is CBD.

17. The liposomal composition of claim 15, wherein the proteolytic enzyme component includes one or both of papain or bromelain.

18. The liposomal composition of claim 15, wherein the phytochemical component includes piceatannol.

19. The liposomal composition of claim 15, further comprising one or both of citrus oil or oregano oil.

20. A liposomal composition, comprising:
cannabidiol;
a proteolytic enzyme component comprising one or both of papain or bromelain;
a phytochemical component comprising piceatannol; and
a plurality of liposomes encapsulating the cannabidiol, proteolytic enzyme component, and phytochemical component.
